(19) United States
(12) Patent Application Publication
(10) Pub. No.: US 2006/0293645 A1
(43) Pub. Date: Dec. 28, 2006

(54) Transilluminating rod used at laparoscopic hysterectomy

(76) Inventor: Michael Cezary Hibner, Phoenix, AZ (US)

Correspondence Address: Michael C. Hibner, 22226 N. 41st Street, Phoenix, AZ, 85050, US

(21) Appl. No.: 11/158,195

(22) Filed: Jun. 22, 2005

(51) Int. Cl. A61B 18/18

(52) U.S. Cl. 606/13; 606/2

(57) ABSTRACT

A transilluminating rod used in laparoscopic hysterectomy has a cylindrical body made of light transmitting material like clear acrylic resin, substantially spherically shaped at the proximal end. An end terminal of predetermined shape and size to match an output connector of an external light source is disposed at the distal end of the rod. When inserted into the vaginal tract of a patient and firmly pressed against vaginal end wall the transilluminating rod brings into prominence a transilluminated and marked out fornix observed from inside of the body cavity through an endoscope. This allows for an easy identification of a cervico-vaginal junction enhancing prospects of safe dissection of a uterus.

### CROSS REFERENCE TO RELATED APPLICATIONS

This application claims the benefit of PPA Ser. No. 60/578,358 filed Jun. 25, 2004 by Michael C. Hibner

### FEDERALLY SPONSORED RESEARCH

Not applicable

### SEQUENCE LISTING

None

### FIELD OF THE INVENTION

This invention relates to instruments used in laparoscopic surgery, specifically to instruments used to identify a cervico-vaginal junction of a patient during a total laparoscopic hysterectomy.

### BACKGROUNG OF THE INVENTION

In recent years, laparoscopy has become one of the foremost procedures in abdominal surgery, resulting decreased blood loss, infection rates as well as aesthetically pleasing effects. Also, the time for healing and consequently the length of patient's hospitalization is greatly reduced. Laparoscopy facilitates performance of a variety of surgical procedures of the abdomen, such as cholecystectomies, appendectomies, hernia repairs, hysterectomies and the like without requiring large incisions or the invasive procedures of conventional surgical techniques.

The present invention is aimed specifically at providing an instrument to facilitate the technique of a total hysterectomy which is removal of the uterus. The main problem associated with that kind of laparoscopic surgical procedure is a reliable identification of the fornix as seen from inside of the body cavity.

Many attempts were made to design and build instruments which would serve that purpose yet those instruments are not very common; in fact, there is not a single one that would be universally adopted and used by the surgeons. However, some inventions in this field might be considered as a related art to the present invention and will be briefly discussed.

U.S. Pat. No. 6,572,631 discloses a transvaginal tube, which can be used at laparoscopic hysterectomy or other laparoscopic surgery involving insertion of the tube into vaginal tract of a patient. The proximal end of the tube is beveled and also angled to receive the cervix. The tube can be made of a transparent material to expose the vaginal epithelium through its walls. There is also a system of sealed ports and valves at the distal end of the tube to secure the pneumoperitoneum inside the body cavity. Among other purposes, an intra-abdominal tissue can be removed through these ports.

U.S. Pat. No. 6,174,317 discloses an instrument for use in uterine laparoscopic surgery. Again, the body of the instrument has a form of an open tube; the proximal end of which is shaped to support against the top wall of the vagina. The instrument includes end closure means detachably fastened to the distal end of the tube; also, the inventors provided incisions at this end to anchor the suture. To maintain the pneumoperitoneum a sealing ring is provided between the tube and the wall of the vagina.

U.S. Pat. No. 5,840,077 discloses an instrument with which a uterus can be manipulated during the laparoscopic hysterectomy. It includes an elongated shaft with a handle, an inflatable sleeve in the middle part which after being filled with air helps to maintain the pneumoperitoneum in the abdomen, an articulated joint, and finally close to the distal end there is a vaginal extender with a cup engaging patient's vaginal cervix to provide an anatomical landmark and backstop for making an incision at the fornix.

U.S. Pat. No. 5,746,750 discloses an instrument specifically designed for manipulation of the uterus, in particular for laparoscopic complete hysterectomy. It comprises a handle at the proximal end of an elongated shank at distal end of which there is a manipulator probe and a claw to grip the cervix. Manipulation is done by tilting the probe around the pivot of the joint by means of an actuating screw. Also means are provided to seal the vagina in order to maintain the pneumoperitoneum.

U.S. Pat. No. 5,394,863 discloses a light transmitting instrument made of an acrylic resin, used to transilluminate the vaginal fornix during the laparoscopic surgery. The body of the instrument has an elongated stem with a cup at the proximal end to engage the cervix. A light source is attached to the distal end of the instrument by means of a light cable. An additional aperture in the elongated body of the instrument can be connected to a suction pump to remove blood or to introduce a catheter when the instrument is used for hydrotubation.

U.S. Pat. No. 5,131,380 discloses an illuminating instrument comprising a transparent semi stiff outer tube with a flexible light conductor inserted into said tube. The combination is intended to illuminate the body's cavities but was not specifically designated for laparoscopic surgery at uterus.

The listed above inventions are cited here as most representative examples only and by no means exhaust the whole lot of instruments patented with the purpose of facilitating the total laparoscopic hysterectomy on mind.

As already mentioned, the main problem associated with laparoscopic hysterectomy is an exact and reliable identification of the fornix and its surroundings as seen from above, that is from the direction of the body cavity. Surgeons skilled in the art of laparoscopic procedures at uterus and particularly the hysterectomy are very well aware of the problems that are awaiting them mainly the danger to cause damage to the bladder or other organs surrounding the uterus.

An instrument or a tool designated to facilitate the hysterectomy should exhibit some few important features.

First of all, it should be so built that when inserted into the patient's vagina it should help to mark out, seen as a kind of a bulge, an exact position of the fornix as observed from the inside of the body cavity through an endoscope. At the same time it should provide enough stretch to the tissues surrounding the cervix in order to make them thinner and to obtain more distance between the uterus and the organs surrounding it. The first two patents listed above that is U.S. Pat. No. 6,572,631 and U.S. Pat. No. 6,174,317 have a tubular shape beveled at the distal end to receive the cervix. When rotated, the tip of the tube is intended to exert pressure on the fornix and produce an imprint observed through an endoscope by the surgeon. U.S. Pat. No. 5,840,077 and U.S. Pat. No. 5,394,863 envelop the cervix with some kind of a cup what means that the pressure on the cervix is uniformly distributed. It should be appreciated, however, that the differences in women's anatomy both as regards the shape and the size of the generative organs preclude using one instrument that would fit all patients in all cases.

Secondly, since the aforementioned feature is not fully sufficient to reliably define the outline of the cervico-vaginal junction some additional means should be employed. As it seems, the easiest and most reliable one is to additionally transilluminate the fornix. This should be facilitated by the fact that the instrument is capable of stretching the fornix so that it becomes more pliable to the penetration of light through it. Out of the above listed and briefly discussed patents only one that is U.S. Pat. No. 5,394,863 is specifically designed to transilluminate the fornix during the procedure of hysterectomy. Yet, as mentioned before, it has a fixed size cup at its distal end what precludes exerting a thrust on a particular part of the fornix. Since an instrument built around this idea has not yet been introduced to the market its usefulness cannot be evaluated.

Also, when the instrument is inserted inside the vagina a proper level of pneumoperitoneum must be maintained during the surgical procedure. Some instruments disclosed in the above mentioned patents do not fulfill that condition in particular the instrument according to U.S. Pat. No. 5,398,863. Some instruments, discerned in other patents, are employing some kind of ports and valves for this purpose.

There are, of course, other considerations that might be taken into account when discussing desirable features of the instrument like, for example, ease of sterilization, ease of handling, cost, etc.

An idea for the invention had been borne in my mind during my fellowship in the Department of Gynecologic Surgery at Mayo Clinic, Scottsdale. There, in some surgical procedures like laparoscopic hysterectomy or colorectal procedures clear acrylic rods, colloquially referred to as Lucite rods (produced by Seelye Plastics), are used. These rods have a common length of 240 mm and different diameters. The biggest diameter is approx. 32 mm and the smallest approx 16 mm. Proximal, by what I mean closer to the patient's body, ends of these rods are shaped like a sphere, distal ends are flat. Inserted into the vagina during the laparoscopic surgery they were meant to stretch and define the position of the fornix during procedures like hysterectomy when decisions had to be made where to cut to dissect the cervix. When the thought to use light to transilluminate the fornix came to my mind I constructed an attachment to the rod comprising a sleeve and a strong flashlight with a four xenon LED light bulbs. The results were astonishingly good and the reliability as well as an ease of hysterectomy procedures I performed using the instrument considerably increased. Despite great improvement there were some drawbacks as well, one that the instrument was % a bit cumbersome as regards sterilization and a special method had to be applied to solve the problem. Also, the power of light source sometimes was not sufficient to contrast with the strong light from an endoscope which had to be switched off during the moment the incision was made.

Later on I improved on the design and adapted the rod so, that an external light source could be directly connected to it. This second prototype is still under tests but preliminary results surpassed my expectations and I find its usefulness invaluable.

Based on experience gathered during testing of my prototypes the objective of the present invention is to provide an instrument which would accomplish the following:
a) afford possibilities for stretching and marking out of the fornix for the purpose of clearly outlying position of the cervico-vaginal junction as well as securing more space between the uterus and the organs surrounding it,
b) ensure strong transillumination of the fornix from outside to make it distinctly seen from inside of the of the patient's body cavity even in a presence of light from an endoscope,
c) make use of commercially available endoscopic light sources,
d) ensure that the pneumoperitoneum is maintained during the surgical procedure,
e) enable easy sterilization and maintenance,
f) make handling of the instrument easy and the price affordable.

### SUMMARY OF THE INVENTION

In accordance with the present invention light from an auxiliary external source is provided to the distal end of cylindrically shaped transparent acrylic rod in order to identify and mark out position of a cervico-vaginal junction of a patient by the way of stretching and transilluminating a vaginal fornix during the laparoscopic total hysterectomy.

### BRIEF DESCRIPTION OF THE DRAWINS

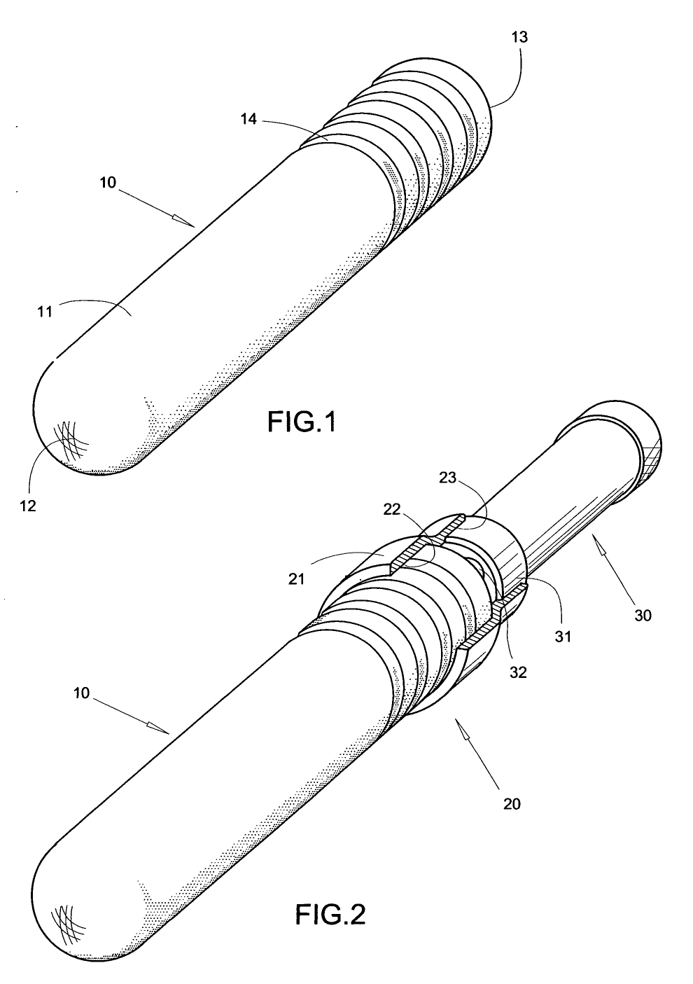

FIG. 1 is an oblique view of a clear acrylic rod used at Mayo Clinic being a Prior Art to the invention FIG. 2 is an oblique view of a first prototype of the instrument produced by the inventor.

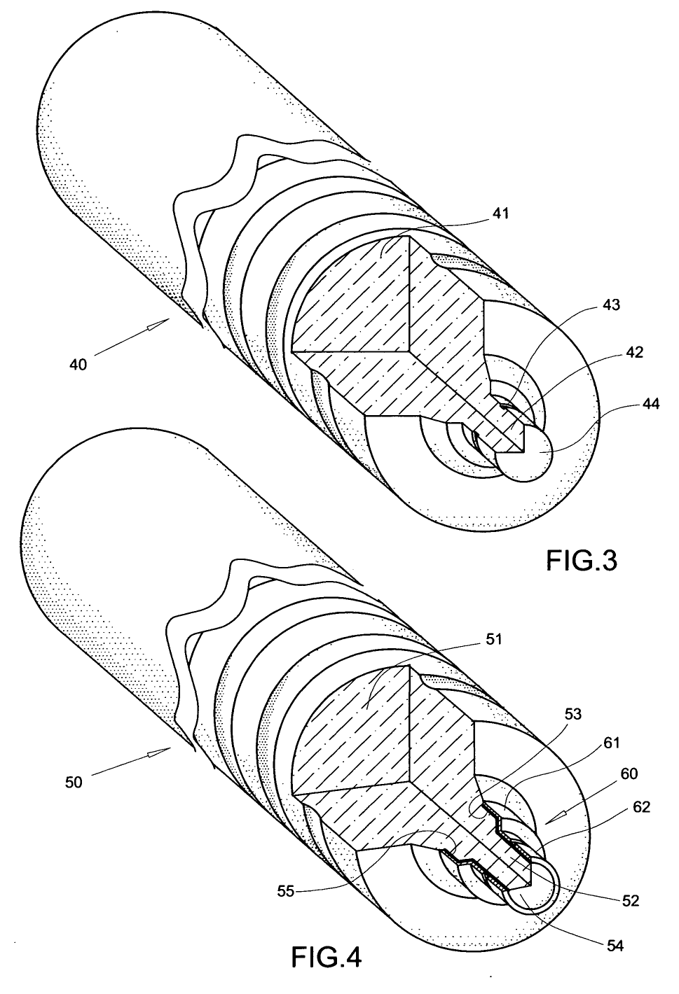

FIGS. 3 and 4 are oblique views of transilluminating rods with integral end terminals.

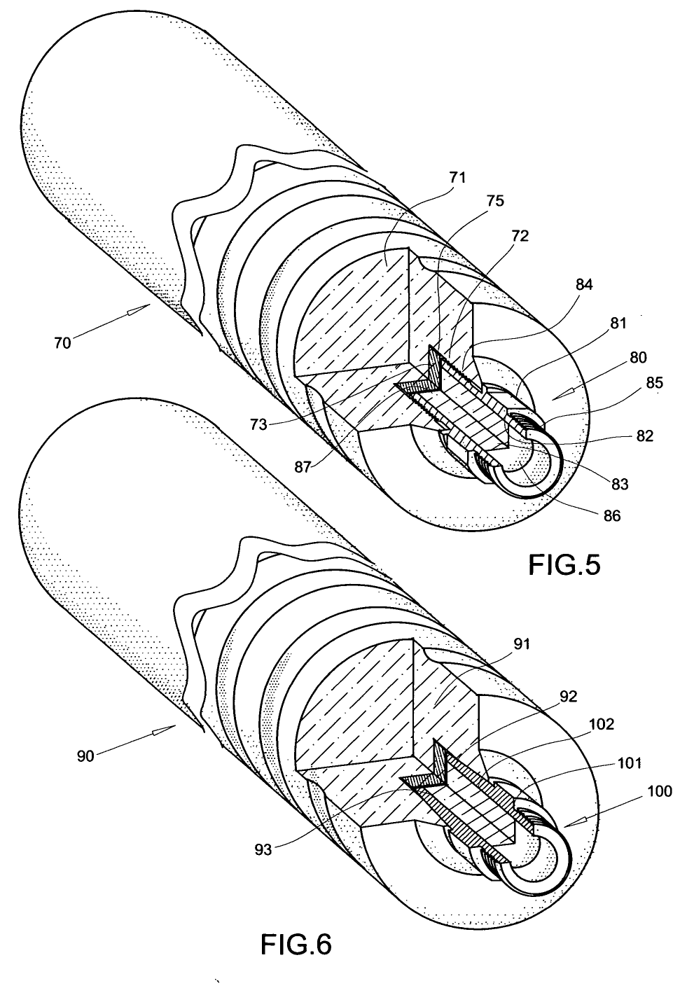

FIGS. 5 and 6 are oblique views of transilluminating rods with detachable end connectors.

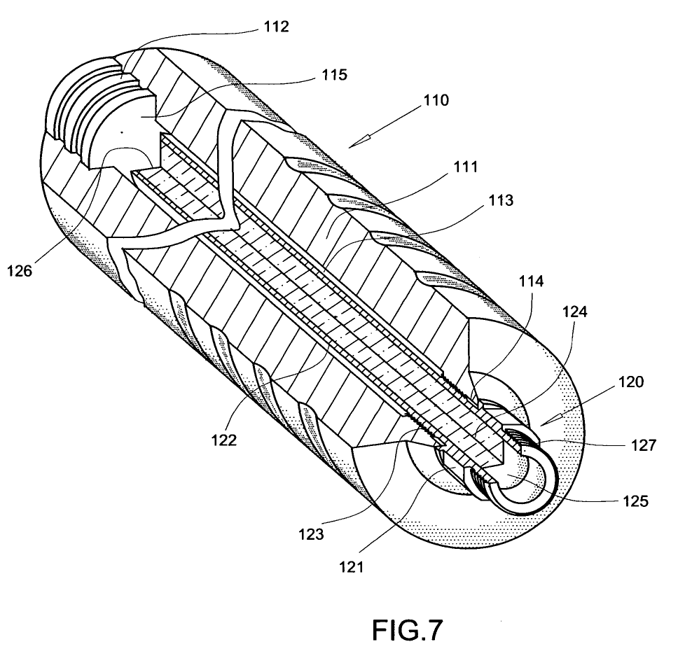

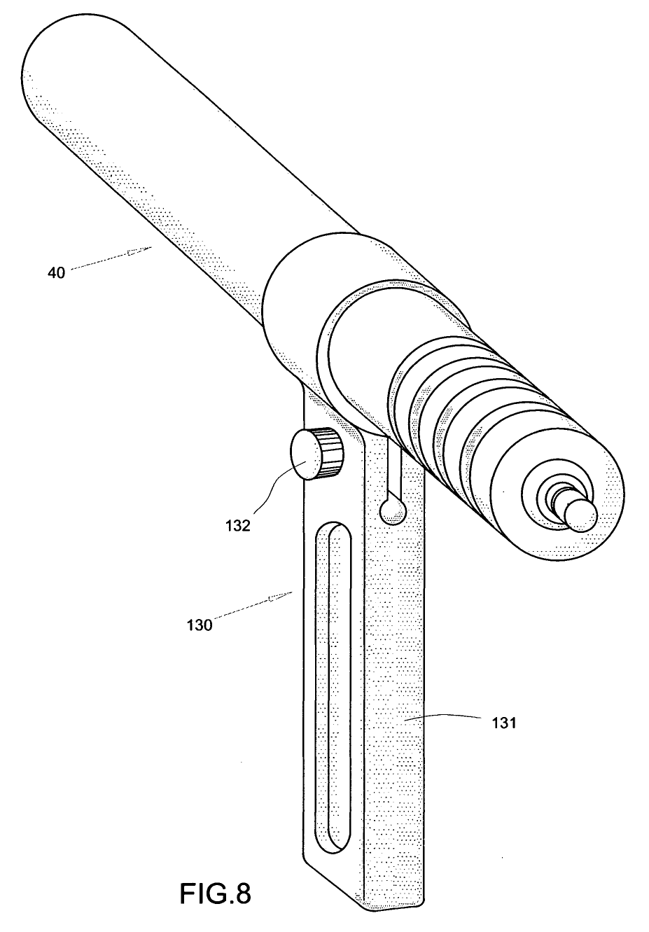

FIG. 7 is an oblique view of an instrument with opaque main body.

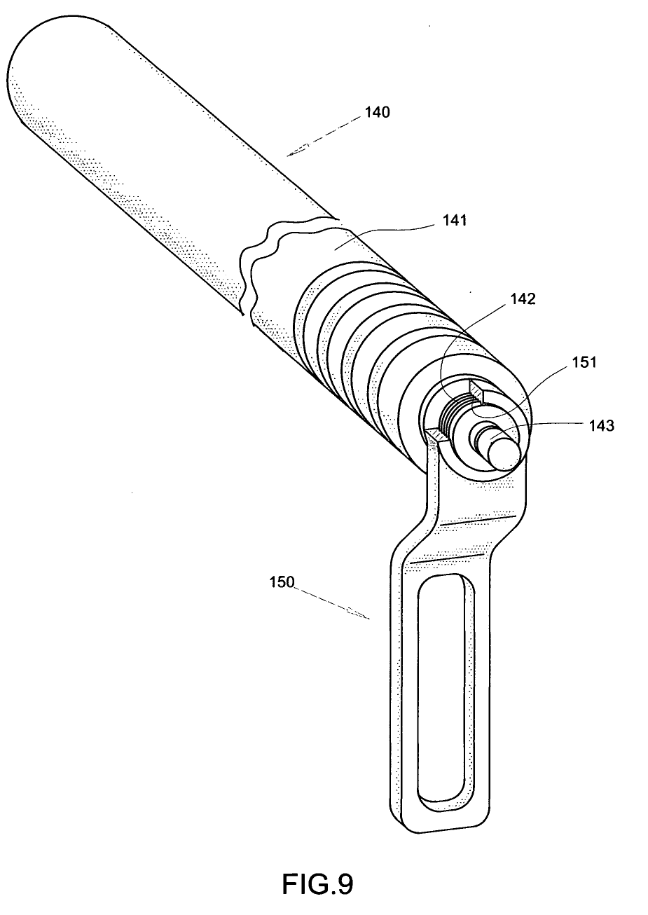

FIG. 8 is an oblique view of a transilluminating rod with slide-over handle attached.

FIG. 9 is an oblique view of a transilluminating rod with a screw-on handle attached.

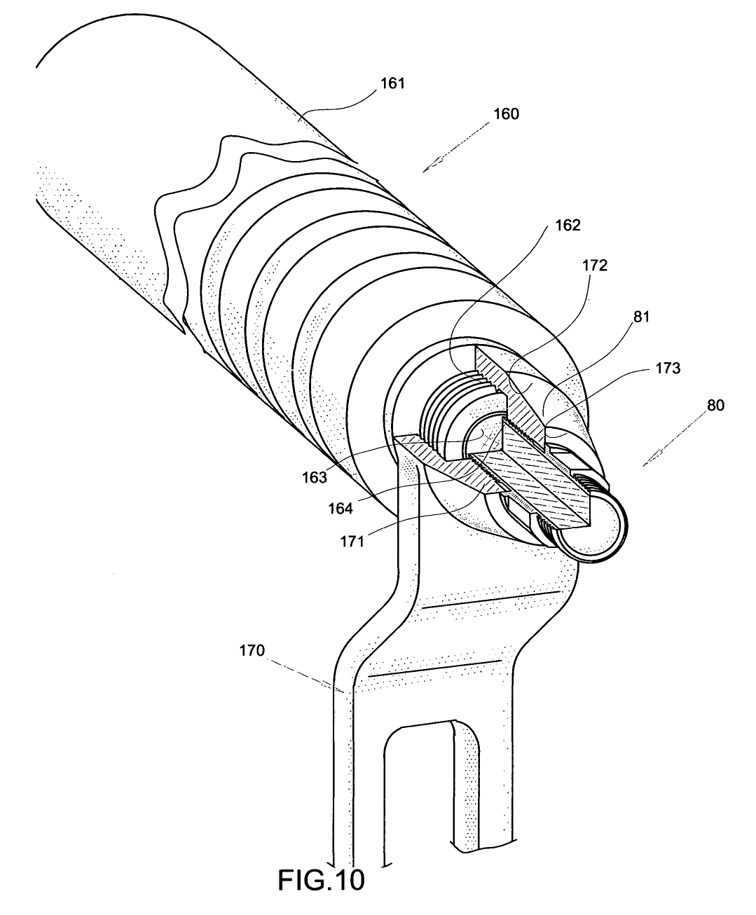

FIG. 10 is an oblique view of another embodiment of a transilluminating rod with a screw-on handle attached.

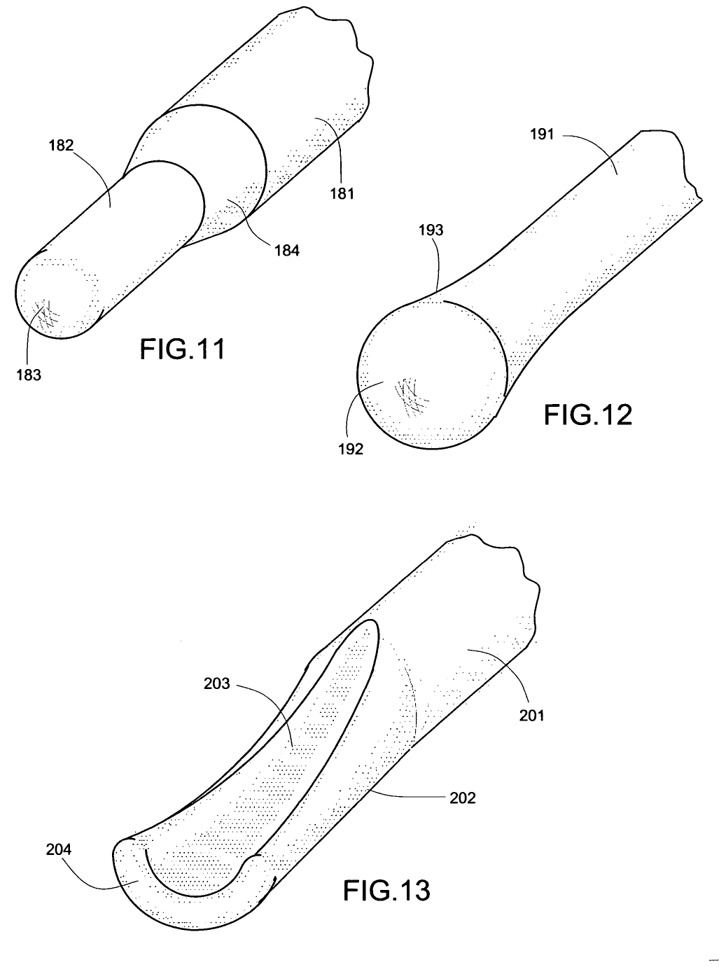

FIGS. 11, 12 and 13 are oblique views of possible alternative shapes of proximal ends of the rods.

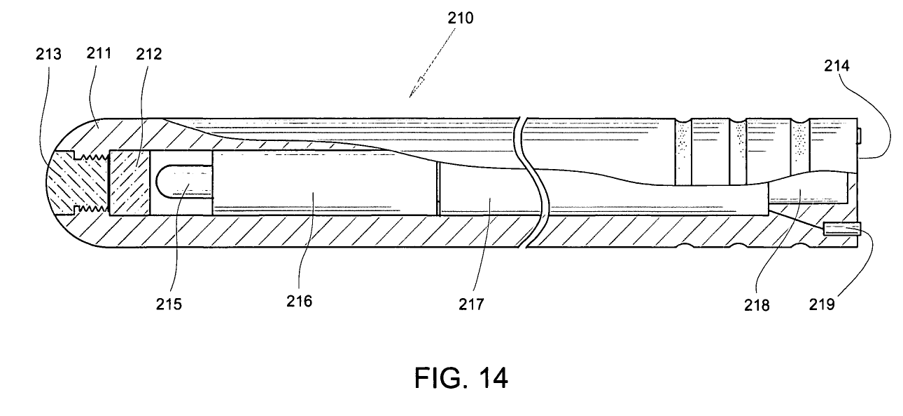

FIG. 14 is a plan view of yet another embodiment of the transilluminating rod.

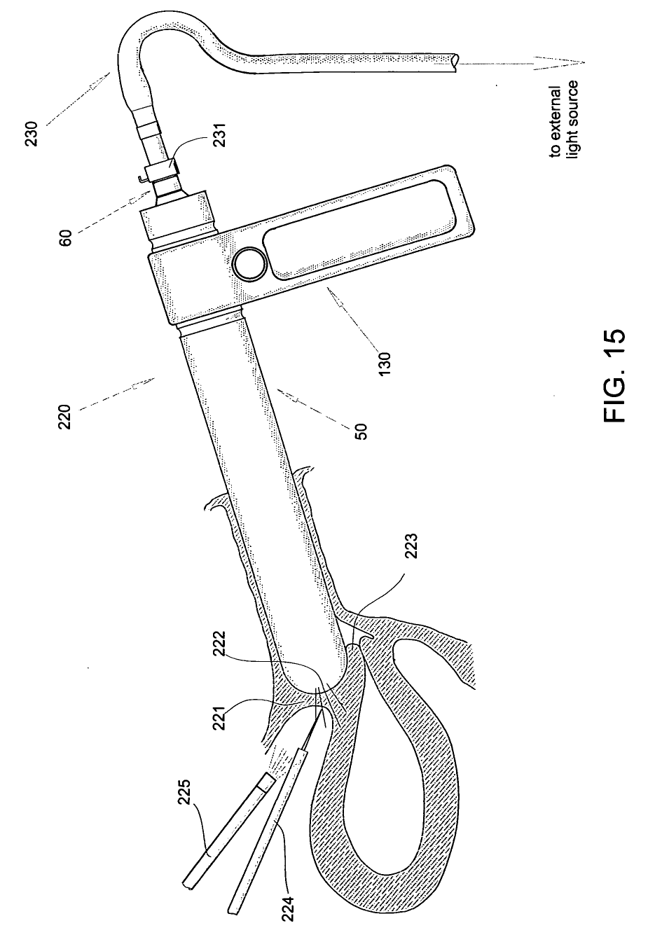

FIG. 15 is a simplified side elevation sectional view of a woman's body showing the instrument in situ.

### DETAILED DESCRIPTION OF THE INVENTION

FIG. 1 shows an original rod 10 used at Mayo Clinic. It is made of transparent acrylic resin LUCITE with cylindrical body 11 spherically ended at proximal end 12 and as a flat plane at distal end 13. There are four grooves 14 close to the distal end to obtain a more secure grip on the rod.

FIG. 2 shows the first prototype of the transilluminating rod I built. It comprises rod 10 depicted in FIG. 1., sleeve 21 and commercially available power torch 30 with four xenon light emitting diodes 32. Sleeve 21 was made from an adapted PVC plumbing socket. There is a transition fit between the outer diameter of rod 10 and inside diameter 22 of sleeve 21 and the same kind of fit between inside diameter 23 of the sleeve and head 31 of flashlight 30.

FIGS. 3, 4, 5 and 6 show four concepts of feeding light from an auxiliary endoscope light source to the distal ends of transilluminating rods. All rods shown in the drawings are made of clear acrylic resin LUCITE, have cylindrical bodies like the original rod 10 and are spherically shaped at their proximal ends. Preferably, they also have a plurality of grooves at the distal end. It will be apparent, however, from drawings 11, 12 and 13 that proximal ends of the rods do not necessarily have to follow spherical shapes but can assume other forms as well. All transilluminating rods of my invention will have lengths in the range 300 to 450 mm, preferably 400 mm and diameters from 18 to 45 mm, preferably 35 mm.

FIG. 3 shows a transilluminating rod 40 to which light from an endoscope cable will be fed to the distal end of the rod through end terminal 42 manufactured as one piece with cylindrical main body 41. Terminal 42 has a form of a stud whose diameter, length and eventually position of groove 43, if any, is such that it will match the type of a light cable output connector attached to it. End face 44 of stud 42 will be cleaved and polished. In the example shown the light cable output connector would be a DYONICS CLIP used, among others, in a STRYKER endoscope cable. This is a simplest form of a transilluminating rod, yet it has some drawbacks as well. One is that the stud can easily be broken off by careless handling. Besides, there are some types of endoscope cables with screw-on-terminals like for example a socket with an internal thread met, among others, in OLYMPUS endoscopes. To cut a fine pitch thread on a stud in a comparatively soft and brittle material would not be practicable if not impossible.

FIG. 4 shows transilluminating rod 50, which is my preferred embodiment and an idea of which was realized in my second prototype. The end terminal has a form of metal ferrule 60, preferably produced of stainless steel. The ferrule is placed on stud 52 being an integral part with main body 51 of the rod, and secured in place with adhesive 55. Stud 52 has an increased diameter 53 closer to main body 51 to match enlarged diameter of hub 61 of the ferrule. This makes the end terminal more robust and less pliable to breaking off. Different types of sleeves can be applied depending on the type of endoscope light sources available. In the example shown, ferrule 60 would match the STRYKER light cable output connector. Face 54 of the terminal is cleaved and polished. FIG. 5 shows transilluminating rod 70 with detachable end connector 80. Recessed hole 72 with an inner thread is made at the distal end of main body 71 of the rod. End connector 80 has a form of sleeve 81 with hole 82 passing through its length. Light guide 83 is inserted and secured inside hole 82 with an adhesive. Light guide 83 can be either made as a solid rod from the clear acrylic resin or, alternatively, a standard light conducting fiberoptic bundle can be used. Thread 84 on one end of sleeve 81 is made to match corresponding thread in recess hole 72. The second end of sleeve 81 is an end terminal to receive an appropriate light cable output connector. The drawing shows thread 85 at the end of sleeve 81 matching, for example, an OLYMPUS endoscope cable. Both ends 86 and 87 of light guide 83 are cleaved and polished. Also, the bottom face 73 of hole 72 will be finished in the same way. Hole 72 will preferably be made deeper to accommodate filter 75. Changed color of transilluminating light, for example to blue, will contrast with a bright white light coming out from a viewing endoscope and greatly enhance the process of identification of internal organs. Making an end terminal on the tool detachable has certain advantages.
(i) end connectors of different types can be used interchangeably with the same main body of the transilluminating rod.
(ii) when, after few times of being used, inevitable scratches and burns appear, only the main body has to be replaced what may be cheaper than replacing the whole tool.
(iii) a color filter can be easily applied.

FIG. 6 shows transilluminating rod 90 with end connector 100, based on the same principle as depicted in FIG. 5. The difference lays in the method of attaching end connector 100 to main body 91 of the rod. Recessed hole 92, shaped like cone on part of its length, is made at the distal end of main body 91 of the rod. Sleeve 101 of end connector 100 also includes cone 102 matching the cone in hole 92. Angles on the cones are such that the connection is self-locking. To disconnect the main body of the rod from the end terminal a special wedge-like prizing tool will be used. Filter 93 can also be placed at the bottom of hole 92. Apart from the method of coupling, end connector 100 is made exactly in the same way as end connector 80 of FIG. 5. An advantage of this method can be that a smooth conical hole may be easier to produce and maintain than the threaded one.

All transilluminating rods depicted so far had main bodies made of transparent acrylic resin. Transilluminating rod 110 shown in FIG. 7 has main body 111 made of some kind of a medical grade opaque material. Insert 112 made of quartz glass or some other kind of hard light conducting material is embedded into main body 111 of the rod at the proximal end. Hole 113 with inner thread 114 at the distal end of body 111 is provided. End connector 120 is similar in built to end connector 80 shown in FIG. 5. It has a form of sleeve 121 with thread 123 by which it is connected to body 111. The difference is in the length since sleeve 121 is extended with stem 122 transferring light through the entire length of the rod to the vicinity of end face 115 of insert 112. Light guide 123 is composed of a fiberoptic bundle and has the length depending on the type of end terminal 120. Both ends 125 and 126 of the light guide will be cleaved and polished. The end connector is also depicted here as one using thread 127 but as already mentioned it can be of any type depending on the type of light source being used. There are certain advantages to this embodiment, one, that a cheaper material can be used for the main body of the rod, two, that the tip at the proximal end of the rod is made of a much harder material what extends a life span of the tool.

It has been found out that due to the slippery nature of the rod, even in the presence of grooves like 14 shown in FIG. 1, manipulating the tool in a patient's vagina is sometimes hampered and some kind of a handle would be of assistance.

FIG. 8 shows transilluminating rod 40 with an auxiliary slide-over handle 130 attached. Body 131 of handle 130 will be preferably made of some kind of a cheaper grade plastic material. Screw 132 locks the handle on the tool. Other types of handles of known art can be used, specifically, universal handles capable of engaging rods of different diameters.

FIG. 9 shows transilluminating rod 140 with an auxiliary screw-on handle 150 attached. Threaded cylinder 142 is provided at the distal end of the main body 141 of the rod just before end terminal 143. It will be appreciated that end terminal 143 not necessarily has to be of the type shown in the drawing but can be any of the types described before and shown in drawings FIGS. 3, 4, 5, 6 and 7. Handle 150, preferably made from stainless steel, has threaded hole 151 to match thread on cylinder 142. Threaded cylinder 142 will have the same size in rods of different diameters so that the same handle can be used for all. Transilluminating rod 140 can be also used independently that is with handle 150 detached.

FIG. 10 shows transilluminating rod 160 being my second preferred embodiment. Handle 170 is detachable but is an integral part of the tool. Threaded cylinder 162 is provided, similarly as in transilluminating rod depicted in FIG. 9, at the distal end of main body 161. Cylinder 162 will be preferably recessed on face 163 to accommodate filter 164. Handle 170 made preferably of stainless steel includes hub 171 with two coaxially displaced and mutually connected threaded holes 172 and 173. Thread inside the first of these two holes matches the thread on cylinder 162 by which handle 170 is attached to main body 161. Second threaded hole 173 serves as housing for detachable end connector. Shown in the drawing is connector 80 depicted in FIG. 5. The most important advantage to this embodiment is that all component parts of the tool are interchangeable.

It has to be noted that methods of connections of parts in some embodiments are shown as exemplifications only and should not be construed as limitations to the scope and specificities of these embodiments. Any known in the art of engineering methods of fastening or coupling can be equally applied.

Proximal ends of all transilluminating rods depicted so far had one feature in common namely that they were shaped like a regular sphere. Although this will be a preferred configuration, it will be appreciated that in some instances, depending on anatomical conditions of a patient, type of an operation, etc. other shapes of proximal ends of transilluminating rods can be of advantage.

FIG. 11 shows the two-stepped transilluminating rod with main body 181 reduced in size to cylinder 182 of smaller diameter and ended like a sphere at proximal end 183. There is transition surface 184 between these two which may assume shape of a straight or concave cone.

FIG. 12 shows the proximal end of a transilluminating rod with main body 191 flaring out to form an enlarged head with spherically shaped proximal end 192. Again, the transition surface 193 between these two may assume a conical shape of any type.

In FIG. 13 another possible form of the proximal end of a transilluminating rod is shown. Main body 201 of the rod makes a transition through surfaces 202 on the outside and a concave surface 203 on the inside to a bean-shaped proximal end 204.

FIG. 14 shows a different concept of transilluminating rod 210 in which light is not supplied from an external source but is generated inside the tool. It is envisaged that all constituent parts of the tool will be contained inside completely sealed body 211 made from some kind of medical grade material. The tool comprises laser diode 215, electronic circuit 216, rechargeable battery 217 and proximal switch 218 enabling the light to be switched on and off; Terminals 219 sealed in distal end 214 of the rod will be used to recharge the battery. Light conducting plate 212 is hermetically closing the interior of the tool close to the proximal end. Insert 213 made from some kind of light transmitting material like quartz glass can be made detachably fixed at the tip of proximal end of the tool.

FIG. 15 shows a simplified cross-section through woman's body with tool 220 in situ, comprising transilluminating rod 50 with handle 130 attached. Light is delivered to end connector 60 at the distal end of the rod through output connector 231 of light cable 230. Another end of the light cable is connected to an auxiliary endoscope light source not shown in the drawing. Tool 220 is inserted into patient's vagina then pressed firmly against fornix 221 and transilluminated by beam of light 222 coming out from the proximal end of rod 50. Position of cervix 223 is revealed clearly showing the surgeon where to make an incision with schematically marked tool 224. Also, schematically marked is the end of endoscope 225 illuminating the body cavity from inside.

What I claim is:

1. A transilluminating rod used at laparoscopic hysterectomy having a proximal end and a distal end, further including a coupling member at said distal end of said rod by which said rod is connected to an external source of light, whereby said proximal end of said transilluminating rod inserted into vaginal tract of a patient and pressed firmly against vaginal end wall, enables a surgeon to observe and reliably identify from inside of the body cavity an exact position of the fornix and its surrounding organs.

2. The transilluminating rod of claim 1 wherein a body of said rod is made of a clear acrylic resin.

3. The transilluminating rod of claim 1 wherein a filter is provided to change color of transilluminating light.

4. The transilluminating rod of claim 1 wherein said rod has a diameter greater than 18 mm.

5. The transilluminating rod of claim 1 wherein said rod has a length greater than 200 mm.

6. The transilluminating rod of claim 1 wherein grooves are provided close to said distal end of said rod whereby said grooves enhance grip on said rod.

7. The transilluminating rod of claim 1 wherein said proximal end of said rod has a spherical convex shape.

8. The transilluminating rod of claim 1 wherein said proximal end of said rod assumes a non-symmetrical shape.

9. The transilluminating rod of claim 1 wherein said rod has a different diameter on part of length of said rod.

10. The transilluminating rod of claim 1 wherein said coupling member assumes a form of an end terminal of predetermined shape and size to match an output connector of said external light source attached thereto, produced as one piece with a body of said rod.

11. The transilluminating rod of claim 1 wherein said coupling member assumes a form of a ferrule of predetermined shape and size to match an output connector of said external light source attached thereto, inseparably disposed over a stub produced as one piece with said body of said rod.

12. The transilluminating rod of claim 1 wherein said coupling member assumes a form of a separable end connector having an end terminal of predetermined shape and size to match an output connector of said external light source attached thereto, affixed to said distal end of said rod by means of a thread, a cone or other means known in the art.

13. The transilluminating rod of claim 12 wherein a light guide is disposed inside a hole passing through said end connector said light guide having both ends finished in a way known in the art.

14. The transilluminating rod of claim 1 wherein said body of said rod is made of any medical grade material suitable for the purpose, substantially other than the clear acrylic resin.

15. The transilluminating rod of claim 14 wherein an insert made of a light transmitting material is disposed at the tip of said distal end of said rod.

16. The transilluminating rod of claim 1 wherein a detachable handle is provided to assist in handling of said rod.

17. The transilluminating rod of claim 15 wherein said handle is adapted to accommodate an end connector.

18. A transilluminating rod for use at laparoscopic hysterectomy wherein a light source is fully integrated and hermetically sealed into a body of a cylindrically shaped rod of a predetermined size and shape, having a proximal end and a distal end.

19. The transilluminating rod of claim 18 wherein at least said proximal end of said rod is manufactured of a light transmitting material.

20. A method of using a transilluminating rod at hysterectomy surgery comprising:
(a) connecting an auxiliary external light source to said transilluminating rod,
(b) inserting said rod into vaginal tract of a patient and exerting pressure with said rod against vaginal end wall,
(c) observing a transilluminated and marked out fornix from inside of the body cavity through an endoscope,
(d) identifying an exact position of the cervico-vaginal junction, (e) dissecting said cervico-vaginal junction with a tool inserted through a laparoscopic port,
(f) grasping and removing of the uterus outside said body cavity through said vaginal tract,
(g) closing an internal end of said vaginal tract by suturing.
